(19) United States
(12) Patent Application Publication
(10) Pub. No.: US 2015/0309053 A1
(43) Pub. Date: Oct. 29, 2015

(54) TEST METHOD AND TEST KIT FOR PSYCHIATRIC AILMENTS

(71) Applicants: RIKEN, Saitama (JP); NATIONAL UNIVERSITY CORPORATION HAMAMATSU UNIVERSITY SCHOOL OF MEDICINE, Hamamatsu-shi, Shizuoka (JP)

(72) Inventors: Takeo Yoshikawa, Saitama (JP); Motoko Maekawa, Saitama (JP); Norio Mori, Shizuoka (JP); Hideo Matsuzaki, Shizuoka (JP); Kazuhiko Nakamura, Shizuoka (JP)

(73) Assignees: RIKEN, Saitama (JP); NATIONAL UNIVERSITY CORPORATION HAMAMATSU UNIVERSITY SCHOOL OF MEDICINE, Hamamatsu-shi, Shizuoka (JP)

(21) Appl. No.: 14/437,771

(22) PCT Filed: Oct. 23, 2013
(86) PCT No.: PCT/JP2013/078703

§ 371 Date: Apr. 22, 2015

(30) Foreign Application Priority Data
Oct. 23, 2012 (JP) 2012-234138

(51) Int. Cl. G01N 33/68

(52) U.S. Cl. CPC G01N 33/6896; G01N 2800/2814; G01N 2800/50; G01N 2333/47

(57) ABSTRACT

A novel test method for testing for the presence or absence of a predisposition to autistic spectrum disorder or the presence or absence of development of autistic spectrum disorder is provided. The test method includes a test step of determining an amount of at least one of the fatty acid binding proteins FABP3, FABP4, FABP5, and FABP7 in a sample prepared from the living body of a human or determining an expression level of at least one of the FABP3, FABP4, FABP5, and FABP7 genes in the sample.

### TECHNICAL FIELD

The present invention relates to a test method and a test kit for mental disorders, especially for autistic spectrum disorder.

### BACKGROUND ART

Autistic spectrum disorder, which is a classified mental disorder, is of obscure etiology. This makes it difficult to identify the disorder by doing tests or detect the disorder early. No method for the curative treatment of autistic spectrum disorder has been established. For autistic spectrum disorder patients to be better socialized, even if only slightly, it is very important for specialists to provide educational intervention (which is called remedial teaching) as early as possible on the basis of early detection. That is, remedial teaching is more effective when started earlier. However, there is no objective and biological criterion for identifying autistic spectrum disorder patients by doing tests. Under such present circumstances, doctors must make autism diagnosis of pediatric patients solely on the basis of the behavior and symptoms of the patients, and it is often very difficult to make early definite diagnosis and detection of autistic spectrum disorder patients.

Incidentally, a possibility has been suggested that abnormal lipid metabolism may be responsible for the mechanism of development of autistic spectrum disorder. For example, in the case of the Smith-Lemli-Opitz syndrome, which is known as an autosomal recessive inherited disorder, a low level of cholesterol and a rise in 7-dehydrocholesterol level occur due to deficiency of 7-dehydrocholesterol reductase, and it has been known that about half of the Smith-Lemli-Opitz syndrome patients are complicated by autistic disorders (Non-patent Literature 1). Further, it has been reported that mice made deficient in 7-dehydrocholesterol reductase exhibit disorders in the serotonin transduction system similar to those which are exhibited by human autistic spectrum disorder patients (Non-patent Literature 2). Furthermore, it has been reported that autistic spectrum disorder patients exhibit changes in amounts of fatty acids in blood plasma or erythrocyte membranes (Non-patent Literature 3). From reports such as these, a possibility has been suggested that lipids may be responsible for the pathological condition of autistic spectrum disorder.

### CITATION LIST

### Non-Patent Literature

### Non-Patent Literature 1

Tierney et al., Am. J. Med. Genet., 98, pp. 191-200, 2001

### Non-Patent Literature 2

Waage-Baudet et al., Int J Dev Neurosci., December; 21(8): pp. 451-459. 2003

### Non-Patent Literature 3

Bell et al., Prostaglandins Leukot Essent Fatty Acids. July-August; 63(1-2): pp. 21-25, 2000

### Non-Patent Literature 4

Sliwinski et al., Neuro Endocrinol Lett., August; 27(4): pp. 465-471, 2006

### Non-Patent Literature 5

Vancassel et al., Prostaglandins Leukot Essent Fatty Acids. July; 65(1): pp. 1-7, 2001

### Non-Patent Literature 6

Wiest et al., Prostaglandins Leukot Essent Fatty Acids. April; 80(4): 221-227, 2009

### Non-Patent Literature 7

Sikora et al., Am. J. Med. Genet., 140A: pp. 1511-1518, 2006

### SUMMARY OF INVENTION

### Technical Problem

Since it is anticipated that lipids and lipid metabolic products, such as cholesterol and fatty acids, per se are low in disease-specificity, it is difficult to use them as molecular markers for autistic spectrum disorder.

The present invention has been made in view of the circumstances described above, and it is an object of the present invention to provide a test method and a test kit for autistic spectrum disorder with use of a novel molecular marker.

### Solution to Problem

As a result of their diligent study to attain the object, the inventors of the present invention found that of the fatty acid binding protein FABP family, amounts of the fatty acid binding proteins FABP3, FABP4, FABP5, and FABP7 in a sample prepared from the living body of a human or expression levels of FABP3, FABP4, FABP5, and FABP7 genes in the sample can be used as molecular markers for autistic spectrum disorder.

That is, the present invention encompasses either of the following inventions:

(1) A test method for testing for the presence or absence of a predisposition to autistic spectrum disorder or the presence or absence of development of autistic spectrum disorder, the test method including a test step of determining an amount of at least one of the fatty acid binding proteins FABP3, FABP4, FABP5, and FABP7 in a sample prepared from the living body of a human or determining an expression level of at least one of the FABP3, FABP4, FABP5, and FABP7 genes in the sample.

(2) A test kit for testing for the presence or absence of a predisposition to autistic spectrum disorder or the presence or absence of development of autistic spectrum disorder, the test kit including a nucleic-acid probe, a nucleic-acid primer, a nucleic-acid aptamer, an antibody, or a peptide probe for detecting at least one of the fatty acid binding proteins FABP3, FABP4, FABP5, and FABP7 in a sample prepared from the living body of a human or at least one of expression products of the FABP3, FABP4, FABP5, and FABP7 genes in the sample.

### Advantageous Effects of Invention

The present invention brings about an effect of providing a test method and a test kit for testing for the presence or absence of a predisposition for autistic spectrum disorder or the presence or absence of development of autistic spectrum disorder.

### BRIEF DESCRIPTION OF DRAWINGS

FIG. 1 shows a graph (upper) showing a concentration of FABP4 in blood plasma in each age group in Example 1 of the present invention and a table (lower) showing the number of children in each age group of autistic spectrum disorder children group and control group.

FIG. 2 is a scatter graph showing a distribution of concentrations of FABP4 in blood plasma in age groups from 6 to 7 years of age in Example 1 of the present invention.

FIG. 3 shows a graph (upper) showing a concentration of FABP4 in blood plasma in each age group in Example 2 of the present invention and a table (lower) showing the number of children in each age group of autistic spectrum disorder children group and healthy control group.

FIG. 4 shows scatter graphs (upper graphs) showing distributions of FABP4 concentrations in blood plasma in age groups from 4 to 6 years of age and age groups from 7 to 8 years of age in Example 2 of the present invention, respectively, and tables (lower tables) showing the results of calculation of the sensitivity, specificity, positive predictive value, and negative predictive value in each of the age groups from 4 to 6 years of age and from 7 to 8 years of age.

### DESCRIPTION OF EMBODIMENTS

Embodiments of the present invention are described below in detail.

[1. Test Method]

A test method according to the present invention is a test method for testing for the presence or absence of a predisposition to autistic spectrum disorder or the presence or absence of development of autistic spectrum disorder, the test method including a test step of determining an amount of at least one of the fatty acid binding proteins FABP3 (fatty acid binding protein 3: sometimes referred to simply as “FABP3”), FABP4 (fatty acid binding protein 4: sometimes referred to simply as “FABP4”), FABP5 (fatty acid binding protein 5: sometimes referred to simply as “FABP5”), and FABP7 (fatty acid binding protein 7: sometimes referred to simply as “FABP7”) in a sample (hereinafter sometimes referred to as “biological sample”) prepared from the living body of a human or determining an expression level of at least one of the FABP3, FABP4, FABP5, and FABP7 genes in the sample.

<Autistic Spectrum Disorder>

The term “autistic spectrum disorder” as used herein specifically encompasses pervasive developmental disorder (PDD), autism, Asperger syndrome, and the like. The symptoms include communication disorder, language developmental disorder, persistence or repetitive behavior, difficulties in interpersonal relationships, and the like.

<Test>

The term “diagnose” or “diagnosis” as used herein refers to the identification of a disorder or a pathological condition by a doctor on the basis of the signs and symptoms of a patient. Meanwhile, the term “testing” or “test” as used herein refers to a test for the presence or absence of a predisposition for autistic spectrum disorder or the presence or absence of development of autistic spectrum disorder in a human being tested (sometimes referred to as “subject”), and does not require a doctor's identification (diagnosis). A test result yielded by the test method of the present invention can be a material for a doctor to make a diagnosis. Further, the test of the present invention can provide important information for example in a case where a compound containing a fatty acid that has a high degree of affinity for a fatty acid binding protein FABP is found and administration of the compound to a human leads to substitution therapy. It should be noted that the concept of “testing for the presence or absence of a predisposition to autistic spectrum disorder” encompasses testing for a possibility of developing autistic spectrum disorder in the future, regardless of whether or not autistic spectrum disorder has already developed. Meanwhile, “testing for the presence or absence of development of autistic spectrum disorder” refers to testing for whether or not autistic spectrum disorder has developed.

<Subjects>

It is more preferable that a subject be younger. For example, in a case where an amount of FABP4 in blood plasma is used as an index, it is preferable that the subject be 8 years old or younger, more preferably 7 years old or younger, even more preferably 6 years old or younger. Receiving the test at an earlier age allows the subject to be early judged as having a predisposition to autistic spectrum disorder or having developed autistic spectrum disorder and, therefore, to receive early remedial teaching.

<Biological Sample>

A biological sample is collected from a subject. The biological sample is not limited to any particular type of biological sample, and needs only contain at least either a protein or a nucleic acid (mRNA as a gene expression product) of the subject. Examples of the biological sample include cell samples, tissue samples, and body fluid samples. Among them, the body fluid samples are preferred. Examples of the body fluid samples include blood samples, lymph samples, spinal fluid samples, and the like, preferably blood samples and lymph samples. Among the blood samples, peripheral blood samples and cord blood samples are especially preferred. A peripheral blood sample can be easily collected, for example, by making a puncture in a fingertip, and therefore poses little burden on the subject. In addition, a peripheral blood sample sufficiently contains a molecular marker that is to be tested by the test method of the present invention. A cord blood sample can be easily collected from the umbilical cord at birth, and has an advantage of making extremely early detection possible.

Further, as a control sample, a biological sample is collected as needed from a healthy subject having no autistic spectrum disorder. It is preferable that the control sample be of the same type as that of the subject (i.e. that collected from the same site). It should be noted that in a case where comparative data is prepared in conducting the test method of the present invention, a biological sample does not need to be collected from a healthy subject.

The biological sample thus collected may be used in testing after being subjected to an operation of extracting a protein or a nucleic acid or an operation of removing an unwanted component, as needed. For example, in a case where a blood sample is used, it is preferable that a blood serum or blood plasma prepared from the collected blood be used in testing.

Further, the biological sample thus obtained may be preserved as needed by a method, such as freeze-preservation, suited to the type of biological sample. By being preserved, the biological sample makes it possible to measure, at a desired time, a molecular marker that is to be tested by the test method. Further, as for preservation, a sample having just been collected may be preserved, or a sample (such as a blood serum or blood plasma) prepared after being collected may be preserved.

<Test Step>

The test method of the present invention includes the following test step (a) or (b):

(a) of determining an amount of at least one of the fatty acid binding proteins FABP3, FABP4, FABP5, and FABP7 in a sample prepared from the living body of a human; or

(b) of determining an expression level of at least one of the FABP3, FABP4, FABP5, and FABP7 genes in the sample.

Whether to use the step (a) or (b) is chosen according to conditions such as the type of biological sample or the type of subject (age, disorder to be tested for). However, the step (a) is preferred. In the case of the step (a), it is preferable that the amount of FABP4 be determined. In the case of the step (b), it is preferable that the expression level of FABP4 be determined.

Step (a)

The step (a) is a step of measuring an amount of at least one of the fatty acid binding proteins FABP3, FABP4, FABP5, and FABP7 in the sample, more specifically an amount of a protein (e.g. a concentration of a protein) that is contained per unit amount of the biological sample. Note, however, that the concept of “measuring an amount of a protein that is contained per unit amount of the biological sample” encompasses both quantitative measurement and qualitative measurement, and encompasses presenting an amount of a protein in a form that can be compared against the control, as well as concentration measurement. More specifically, for example, the concept also encompasses comparing data at the time of acquisition prior to conversion into a concentration with use of a calibration curve, presenting a result in a form that shows whether or not an amount of a protein exceeds a certain threshold value, and the like.

The amount of at least one of the proteins FABP3, FABP4, FABP5, and FABP7 may be measured by any method. Examples of such a method include: a method that involves the use of an immunological technique that involves the use of an antibody specific for FABP3, FABP4, FABP5, or FABP7; liquid column chromatography; mass spectrometry; and the like. Examples of the method that involves the use of an antibody include ELISA (enzyme-linked immuno sorbent assay), a quantitative western blotting method, and immunoprecipitation, and it is preferable that ELISA be used. Examples of types of ELISA include, but are not particularly limited to: antigen-measuring types of ELISA (to measure the amount of an antigen that is contained in a biological sample), such as ELISA based on a direct adsorption method, ELISA based on a competition method, and ELISA based on a sandwich method; ELISA specialized in measuring a minute amount of a sample, such as microchannel types of ELISA or ELISA that involves the use of microbeads; and the like.

The antibody specific for FABP3, FABP4, FABP5, or FABP7 may be a monoclonal antibody or a polyclonal antibody, but it is preferable that the antibody specific for FABP3, FABP4, FABP5, or FABP7 be a polyclonal antibody. For example, the amino acid sequences of human FABP3, FABP4, FABP5, and FABP7 are available from a public database such as NCBI. For example, in the NCBI database, the accession number of FABP4 is CAG33184/NP_{—}001433. An example of FABP4 has the amino acid sequence of SEQ ID NO. 1. Based on this information, a person skilled in the art can easily determine appropriate amino acid sequences as antigens for constructing antibodies specific for FABP3, FABP4, FABP5, and FABP7.

In the present invention, the “antibody” is intended to be in a form that encompasses all classes and subclasses of immunoglobulin and a functional fragment of the antibody. The concept “antibody” encompasses a natural antibody, whether polyclonal or monoclonal. In addition, this concept encompasses an antibody that is produced by gene recombination technology and a functional fragment of such an antibody. The term “functional fragment of the antibody” refers to a fragment that has a partial region of the aforementioned antibody and has an antigen-binding capacity (synonymous with a binding fragment). The natural antibody may be derived from, but is not particularly limited to, any of the various species of organism such as humans, mice, rats, goats, rabbits, camels, horses, cows, chickens, sharks, and fish. Examples of the antibody that is produced by gene recombination technology include, but are not particularly limited to: chimeric antibodies, such as humanized antibodies and primatized antibodies, that are obtained through genetic modification of natural antibodies; synthetic antibodies; recombinant antibodies; mutation-introduced antibodies; grafted antibodies (e.g. antibodies with which other proteins, radioactive labels, or the like are conjugated or fused); and antibodies obtained by subjecting antibodies already produced by gene recombination technology to modification that is similar to the aforementioned genetic modification of natural antibodies. Further, specific examples of the functional fragment of the antibody include F(ab′)₂, Fab′, Fab, Fv (variable fragment of antibody), sFv, dsFv (disulphide stabilized Fv), dAb (single domain antibody), and the like (George et al, Exp. Opin. Ther. Patents, Vol. 6, No. 5, p. 441-456, 1996).

Furthermore, the concept “binding fragment” in the present invention encompasses an antibody fragment mutation-introduced within such limits as to maintain reactivity to the target protein. The aforementioned mutation is performed by a publicly-known technique, such as a genetic modification technique, selected as appropriate by a person skilled in the art.

Step (b)

The step (b) is a step of measuring, with use of the biological sample, an expression level of at least one of FABP3, FABP4, FABP5, and FABP7 genes in the sample. It should be noted that the FABP3 gene is a general term for a nucleic acid encoding FABP3, that the FABP4 gene is a general term for a nucleic acid encoding FABP4, that the FABP5 gene is a general term for a nucleic acid encoding FABP5, and that the FABP7 gene is a general term for a nucleic acid encoding FABP7.

The expression level of at least one of the FABP3, FABP4, FABP5, and FABP7 genes may be measured by any method, but may be measured by a method including a technique for amplifying a desired nucleic acid (e.g. mRNA as a transcription product) with a nucleic acid amplification technique such as PCR. An example of a method that involves the use of a nucleic acid amplification technique is quantitative RT-PCR, and an example of a method for the direct detection of mRNA is Northern blot or the like. Alternatively, the method for measuring the expression level of the gene may involve the use of a nucleic-acid chip such as a microarray. The term “measuring the expression level of the gene” can be used interchangeably with “measuring an amount (such as a concentration) of an expression product (which will be described later) of the gene”.

In the measurement of the expression level of the gene, cDNA may be prepared by using, as a template, the mRAN contained in the biological sample. Amplification of the FABP3, FABP4, FABP5, and FABP7 genes as the mRNA can be performed on the basis of nucleotide sequence information available from a public database such as NCBI. For example, in the NCBI database, the accession number of the FABP4 gene is NM_{—}001442. An example of the FABP4 gene has the nucleotide sequence of SEQ ID NO. 2. Based on this information, a person skilled in the art can easily design an appropriate primer for amplifying the FABP3, FABP4, FABP5, or FABP7 gene.

<Determination>

Determination of the presence or absence of a predisposition to autistic spectrum disorder or the presence or absence of development of autistic spectrum disorder is made by obtaining a protein amount of at least one of FABP3, FABP4, FABP5, and FABP7 or an expression level of at least one of the FABP3, FABP4, FABP5, and FABP7 genes in the aforementioned test step (a) or (b) and comparing the protein amount or expression level obtained from the subject against that obtained from the control.

In one example of determination, a subject whose biological sample is significantly lower in amount of at least one of FABP3, FABP4, FABP5, and FABP7 (whether a protein amount or a gene expression level) than that of the control is judged as having a predisposition to autistic spectrum disorder or having developed autistic spectrum disorder. The concept “significantly lower (smaller) in amount” here encompasses a comparison between specific numerical values and a comparison between relative amounts (it is not necessary to perform actual calculations of amounts, but it is judged whether an amount is higher or lower than a certain reference amount), regardless of whether such amounts are results of quantitative measurement or results of qualitative measurement.

The control sample may be conducted at the same time as or at a different time from the time when the test of the sample from the subject is conducted. That is, the numerical value of the control sample that is compared against the numerical value of the subject may be a value obtained through a test conducted at a time different from the time when the sample from the subject is tested. Further, the test of the control sample does not need to be conducted by a person who conducts the test of the subject. For example, a control sample test value already obtained and stored in a database or the like may be used as a threshold value.

As a numerical value(s) of a control sample(s) for use in determination, it is possible to directly use a numerical value of an individual healthy subject or to use a mean value obtained from a population of numerical values of a certain number of healthy subjects. Alternatively, it is possible to set a cutoff value in advance and compare the numerical value obtained from the subject against the cutoff value. For example, in a case where a protein amount of at least one of FABP3, FABP4, FABP5, and FABP7 in a biological sample from a subject or an expression level of at least one of the FABP3, FABP4, FABP5, and FABP7 genes in the sample is equal to or larger than the cutoff value, the subject can be judged as having a low possibility of developing autistic spectrum disorder. On the other hand, in a case where a protein amount of at least one of FABP3, FABP4, FABP5, and FABP7 in a sample prepared from a subject or an expression level of at least one of the FABP3, FABP4, FABP5, and FABP7 genes in the sample is lower than the cutoff value, the subject can be judged as having a possibility and risk of developing autistic spectrum disorder.

The term “cutoff value” refers to such a value at which both diagnostic sensitivity (true positive rate) and diagnostic specificity (true negative rate) are sufficiently high in a case where the presence or absence of a predisposition to a disease or the presence or absence of the disease is determined with reference to that value. For example, it is possible to set, as the cutoff value, a value at which individuals having developed autistic spectrum disorder exhibit a high positive rate and individuals free of autistic spectrum disorder exhibit a high negative rate.

The term “diagnostic sensitivity” here refers to the percentage (true positive percentage) of positive results (abnormal values) obtained through a test conducted on a group of subjects having a predisposition to a particular disease or having a particular disease. The term “diagnostic specificity” here refers to the percentage (true negative percentage) of negative results (normal values) obtained through a test conducted on a group of subjects not suffering from any particular disease. Further, the term “positive predictive value” refers to the percentage of individuals who, of those subjects tested positive as a result of the test, are actually suffering from the disease, and the term “negative predictive value” refers to the percentage of individuals who, of those subjects tested negative as a result of the test, are actually not suffering from the disease.

The cutoff value can be calculated by a method that is publicly known in the technical field to which the present invention pertains. For example, protein amounts of at least one of the fatty acid binding proteins FABP3, FABP4, FABP5, and FABP7 in a sample prepared from an individual developing autistic spectrum disorder and in a sample prepared from an individual free of autistic spectrum disorder or expression levels of at least one of the FABP3, FABP4, FABP5, and FABP7 genes in the samples are calculated, and diagnostic sensitivity and diagnostic specificity at the values thus calculated are obtained. On the basis of the values thus obtained, an ROC (receiver operating characteristic) curve is created using suitable commercially-available analysis software. Then, a value at which the diagnostic sensitivity and the diagnostic specificity are as close as possible to 100% is obtained from the curve, and the value can be used as the cutoff value. Alternatively, it is also preferable to use, as the cutoff value, a “mean value+2 standard deviations” of protein amounts of at least one of the fatty acid binding proteins FABP3, FABP4, FABP5, and FABP7 in samples prepared from a large number of healthy subjects or expression levels of at least one of the FABP3, FABP4, FABP5, and FABP7 genes in the samples. Use of this value makes it possible to determine the presence of or the risk of development of autistic spectrum disorder with high sensitivity and specificity.

For example, in an example shown in Example 1, it is demonstrated that all risks of developing autistic spectrum disorder, i.e. the presence or absence of a predisposition to autistic spectrum disorder or the presence or absence of development of autistic spectrum disorder, can be detected by using, as the cutoff value, the mean of numerical values (protein concentrations) obtained from a plurality of control samples or a value that is slightly lower than the mean (i.e. 15 ng/ml to 16 ng/ml). The cutoff value may alternatively be determined by classifying data according to age and creating an ROC curve for each age.

[2. Test Kit]

Further, the present invention provides a test kit for testing for the presence or absence of a predisposition to autistic spectrum disorder or the presence or absence of development of autistic spectrum disorder, the test kit including a nucleic-acid probe, a nucleic-acid primer, a nucleic-acid aptamer, an antibody, or a peptide probe for detecting at least one of the fatty acid binding proteins FABP3, FABP4, FABP5, and FABP7 in a sample prepared from the living body of a human or at least one of expression products of the FABP3, FABP4, FABP5, and FABP7 genes in the sample.

The term “expression product” here refers to an mRNA transcribed from the FABP3 gene, the FABP4 gene, the FABP5 gene, or the FABP7 gene. The test kit of the present invention encompasses a form of detection of cDNA obtained through reverse transcription of the mRNA.

The “nucleic-acid probe” refers to a nucleic-acid probe that binds specifically to any of the expression products, and more specific examples thereof include a TaqMan probe, an Invador probe, and the like. The “nucleic-acid primer” refers to a nucleic-acid primer capable of specifically amplifying an mRAN as the expression product or cDNA obtained through reverse transcription of the mRNA, and a more specific example is a primer that is used in a nucleic acid amplification method such as RT-PCR. The “nucleic-acid aptamer” refers to a nucleic-acid construct constituted by a nucleic acid that binds specifically to any of the fatty acid binding proteins FABP3, FABP4, FABP5, and FABP7 contained in the biological sample.

The “peptide probe” refers to a peptidic probe that binds specifically to any of the fatty acid binding proteins FABP3, FABP4, FABP5, and FABP7. A specific example is a peptide sequence that binds specifically to FABP3, FABP4, FABP5, or FABP7.

The nucleic-acid probe, nucleic-acid primer, and nucleic-acid aptamer of the kit may be constituted by including a non-natural nucleic acid (such as PNA) as well as a natural nucleic acid. Similarly, the peptide probe may also be constituted by including a non-natural amino acid as well as a natural amino acid.

The test kit according to the present invention may further include as needed at least one of the following: various reagents and instruments (a polymerase, a PCR buffer, each dNTP, a pipette, etc.) for use in a nucleic acid amplification method such as PCR; various reagents and instruments (a test tube, a buffer, etc.) for use in the preparation of a sample; various reagents and instruments (an electrophoretic gel material, a pipette, etc.) for use in the analysis of a nucleic acid amplification fragment; instructions for use of the test kit; a control sample for use in measurement; comparative data for use in the analysis of a measurement result; and the like. It should be noted that the instructions for use of the test kit contains a record of the test method according to the present invention as described above in section [1. Test Method].

As described above, the test method and the test kit of the present invention make it possible to make a diagnosis of autistic spectrum disorder on the basis of a test with the introduction of a biological criterion, whereas the conventional diagnosis of autistic spectrum disorder has depended greatly on the personal opinions of the doctors. Therefore, the test method and the test kit are expected to bring about improvement in diagnostic technology. Further, the test method and the test kit make early detection possible, thus making it possible to effectively provide early remedial teaching to the patients. Further, the test of the present invention can provide important information for example in a case where a compound containing a fatty acid that has a high degree of affinity for a fatty acid binding protein FABP is found and administration of the compound to a human leads to substitution therapy.

[3. Screening of Drugs for Treating Autistic Spectrum Disorder and the Like]

Induced pluripotent stem cells (iPS cells) generated from a human with such a genetic mutation that a stop codon appears in a region encoding the protein FABP4, such as those shown below in Reference Example 1, are useful, for example, as (1) applications of screening of drugs for treating autistic spectrum disorder, (2) disease model cells for the confirmation of the efficacy of a candidate drug for treating autistic spectrum disorder, (3) disease model cells for the elucidation of pathogenic mechanism and pathological condition, and the like.

[4. Use of Test Results Obtained by the Test Method]

Test results obtained by conducting a test method as described above in section [1. Test Method] can be used as a diagnostic material for a doctor to make a diagnosis. As a result of conducting a test method as described above in section [1. Test Method], a subject judged as having a risk of developing autistic spectrum disorder (i.e. as having a predisposition to autistic spectrum disorder) or a subject judged as having developed autistic spectrum disorder can be subjected to treatment based on the result of a doctor's diagnosis as needed. An example of the treatment here is remedial teaching that is provided by a doctor or by an expert other than a doctor.

[5. Example of a System for Executing a Test Method According to the Present Invention]

A test system that is used for executing the aforementioned test method according to the present invention and a test method that involves the use of the test system fall within the scope of the present invention. The present embodiment is described by taking, as an example, a case where members constituting a test system according to an embodiment of the present invention are “functional blocks that are realized by an arithmetic section such as a CPU executing a program code stored in a recording medium such as ROM or RAM”. However, the functional blocks may alternatively be realized by hardware that performs the same processes. Further, the functional blocks can alternatively be realized by a combination of hardware that performs some of the processes and the arithmetic section, which executes a program code for performing control of the hardware and the remaining processes. Furthermore, even those ones of the members which are described as hardware can be realized by a combination of hardware that performs some of the processes and the arithmetic section, which executes a program code for performing control of the hardware and the remaining processes. It should be noted that the arithmetic section may be a single arithmetic section, or a plurality of arithmetic sections connected to each other via buses inside the apparatus and/or various communication paths may execute a program code in cooperation with each other.

An example of a test system according to the present invention includes, as its functional blocks, at least an input receiving section 11A, a measuring section 11, a storage section 12, a CPU 13, and a display section 14. The input receiving section 11A is an interface configured to receive an input. The input receiving section 11A is configured as an interface to connect the test system to an external device. In some cases, the input receiving section 11A is configured as an input device such as a keyboard or a mouse. Conditions for operation of the test system and the like are inputted, for example, through the input receiving section 11A. The measuring section 11 is configured to include: (1) a receiving section in which the aforementioned biological sample collected from a subject is put; and (2) a measuring device configured to produce a signal value for measuring an amount of at least one of the fatty acid binding proteins FABP3, FABP4, FABP5, and FABP7 in the biological sample or measuring an expression level of at least one of the FABP3, FABP4, FABP5, and FABP7 genes in the biological sample. An example of the measuring device is a device configured to produce a signal value for measuring an amount of at least one of the fatty acid binding proteins FABP3, FABP4, FABP5, and FABP7, preferably FABP4, in the biological sample according to ELISA. The storage section 12 includes a measurement value receiving section 12a in which the aforementioned signal value for measuring a concentration of a biomarker such as FABP3 as measured by the measuring device is put. The storage section 12 may further include a reference value storage section 12b (e.g. configured as a memory) in which a reference value (cutoff value) for determination of the presence or absence of a predisposition to autistic spectrum disorder or the presence or absence of development of autistic spectrum disorder is stored. The CPU 13 includes an arithmetic section 13a configured to calculate a concentration value from the aforementioned signal value for measuring a concentration of a biomarker such as FABP3. The CPU 13 further functions as an arithmetic section 13b configured to generate information for determination of the presence or absence of a predisposition to autistic spectrum disorder or the presence or absence of development of autistic spectrum disorder and as a determination section 13c configured to receive evaluation information from the arithmetic section 13b and make the determination. The display section 14 (e.g. configured as a liquid crystal display or a printer) has a function of displaying a measured value obtained through arithmetic processing by the arithmetic section 13a and a function as a determination result display section 14 of displaying a determination result yielded by the determination section 13c. It should be noted that this functional block is realized by the CPU 13 executing a program stored in the storage section 12 and controlling a peripheral circuit such as an input-output circuit.

The following illustrates, as an aspect of the test system, a system for evaluating autistic spectrum disorder by using the fatty acid binding protein FABP4 as a biomarker, and specifically describes a test process. In this test system, Step 1 is executed in which a biological sample is set in the receiving section of the measuring section 11 and the concentration of FABP4 in the biological sample is automatically measured by the measuring device of the measuring section 11. It should be noted that the method for measuring the concentration of FABP4 is as described in detail above in “Step (a)” and “Step (b)” of section <Test Step>. Next, Step 2 is executed in which the measured value receiving section 12a receives (stores) a measured value of the concentration of FABP4 as measured in Step 1. Next, Step 3 is executed in which the arithmetic section 13a obtains a measured value through arithmetic processing from a signal value for measuring the concentration of FABP4 stored in the measured value receiving section 12a and the arithmetic section 13b generates, with reference to the measured value and a reference value (e.g. a cutoff value) stored in the reference value storage section 12b, evaluation information for determining the presence or absence of a predisposition to autistic spectrum disorder or the presence or absence of development of autistic spectrum disorder. Next, Step 4 is executed in which on the basis of the evaluation information generated in Step 3, the determination section 13c determines the presence or absence of a predisposition to autistic spectrum disorder or the presence or absence of development of autistic spectrum disorder. Next, Step 5 is executed in which the display section 14 displays a result of the determination of the presence or absence of a predisposition to autistic spectrum disorder or the presence or absence of development of autistic spectrum disorder as generated by the determination section 13c. It should be noted that the steps from the generation of the evaluation information to the determination (steps 3 to 4) can be executed by operating an evaluation program stored in the storage section 12. The evaluation program makes determination as described above in section <Determination>. More specifically, the evaluation program compares the measured value of the concentration of FABP4 stored in the measured value receiving section 12a against the cutoff value stored in the reference value storage section 12b, and generates a result of the comparison as the evaluation information. Then, in a case where the measured value of the concentration of FABP4 is lower than the cutoff value, the evaluation program determines that the subject has a possibility and risk of developing autistic spectrum disorder.

[6. Examples of Specific Aspects of the Present Invention]

For example, the present invention encompasses any of the following inventions:

(1) A test method for testing for the presence or absence of a predisposition to autistic spectrum disorder or the presence or absence of development of autistic spectrum disorder, the test method including a test step of determining an amount of at least one of the fatty acid binding proteins FABP3, FABP4, FABP5, and FABP7 in a sample prepared from the living body of a human or determining an expression level of at least one of the FABP3, FABP4, FABP5, and FABP7 genes in the sample.

(2) The test method as set forth in (1), wherein in the test step, the amount of the fatty acid binding protein FABP4 or the expression level of the FABP4 gene is determined.

(3) The test method as set forth in (1) or (2), wherein the sample is a blood-derived sample.

(4) The test method as set forth in any one of (1) to (3), wherein the amount of the protein being tested is determined.

(5) The test method as set forth in any one of (1) to (4), wherein the human is 8 years old or younger.

(6) The test method as set forth in any one of (1) to (5), wherein in the test step, the presence of a predisposition to autistic spectrum disorder or the presence of development of autistic spectrum disorder is determined when at least one selected from among the amounts of the fatty acid binding proteins FABP3, FABP4, FABP5, and FABP7 in the sample and the expression levels of the FABP3, FABP4, FABP5, and FABP7 genes in the sample is lower than that of a healthy subject.

(7) The test method as set forth in (6), wherein in the test step, the presence of a predisposition to autistic spectrum disorder or the presence of development of autistic spectrum disorder is determined by using a test system including at least an input receiving section, a measuring section, a storage section, a CPU, and a display section as functional blocks.

(8) The test method as set forth in any one of (1) to (7), wherein the autistic spectrum disorder is autism.

(9) A test kit for testing for the presence or absence of a predisposition to autistic spectrum disorder or the presence or absence of development of autistic spectrum disorder, the test kit including a nucleic-acid probe, a nucleic-acid primer, a nucleic-acid aptamer, an antibody, or a peptide probe for detecting at least one of the fatty acid binding proteins FABP3, FABP4, FABP5, and FABP7 in a sample prepared from the living body of a human or at least one of expression products of the FABP3, FABP4, FABP5, and FABP7 genes in the sample.

### EXAMPLES

The present invention is described in more detail below with reference to a reference example and examples.

### Reference Example 1

For the purpose of searching for candidates for a novel molecular marker for autistic spectrum disorder, a screening of mutations on the FABP4 gene was performed with use of DNA samples from autistic spectrum disorder children. As a result, it was found that one out of 267 family lines of autistic spectrum disorder exhibited a mutation in which the 294th G located at exon 3 of a gene region encoding the protein FABP4 had been replaced with A and, as a result, a codon designating the 98th tryptophan in the amino acid sequence of FABP4 had been replaced with a stop codon.

### Example 1

### Preparation of Biological Samples

A test was done on an autistic children group (Autism (native)) of 116 males from 6 to 19 years of age diagnosed by doctors as having developed autism and a normally-developing children group (control, i.e. a control group) of 127 healthy children from 5 to 19 years of age. The test was done on all of the subjects, and biological samples were prepared from all of the subjects. None of the subjects had been on medication.

From each of these subjects, about 6 cc of peripheral blood was collected by making a peripheral puncture in the median cubital vein, the median radial vein, the dorsal hand vein, or the like. Furthermore, the peripheral blood thus collected was centrifuged to separate the blood serum from the cell precipitate. The blood serum thus obtained had been preserved at −80° C. until it was used in testing.

(Measurement of the Concentration of the Fatty Acid Binding Protein FABP4)

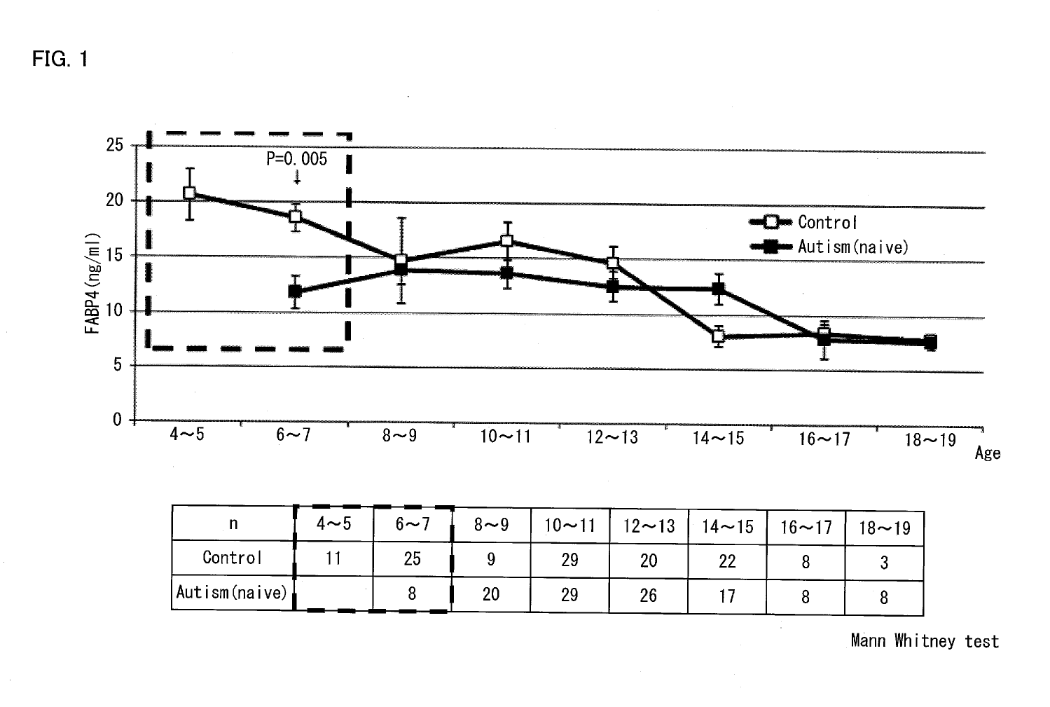

The concentration of the fatty acid binding protein FABP4 in the blood serum thus obtained was measured by ELISA using a commercially available ELISA kit (HUMAN AFABP ENZYME IMMUNOASSAY KIT, SPI bio). The measurements were performed as follows: The samples were prepared by diluting 30 μL of a blood serum of each subject with a buffer solution (EIA buffer, which accompanied the ELISA kit) at a 10-fold dilution factor. Then, the autistic children group and the control group were both divided into age groups from 4 to 5 years of age, from 6 to 7 years of age, from 8 to 9 years of age, from 10 to 11 years of age, from 12 to 13 years of age, from 14 to 15 years of age, from 16 to 17 years of age, and from 18 to 19 years of age, and the mean value of FABP4 protein concentrations (referred to as “mean concentration”) in each age group was calculated. Furthermore, the mean concentrations in the same-age groups (Age) of autistic children and control group children were compared against each other to see if there is a significant difference between the mean concentration in the control group and the mean concentration in the autistic children group. The results are shown in the upper graph of FIG. 1. The number of children in each age group of autistic children and control group children is shown in the lower table of FIG. 1.

(Analysis of the Measurement Results)

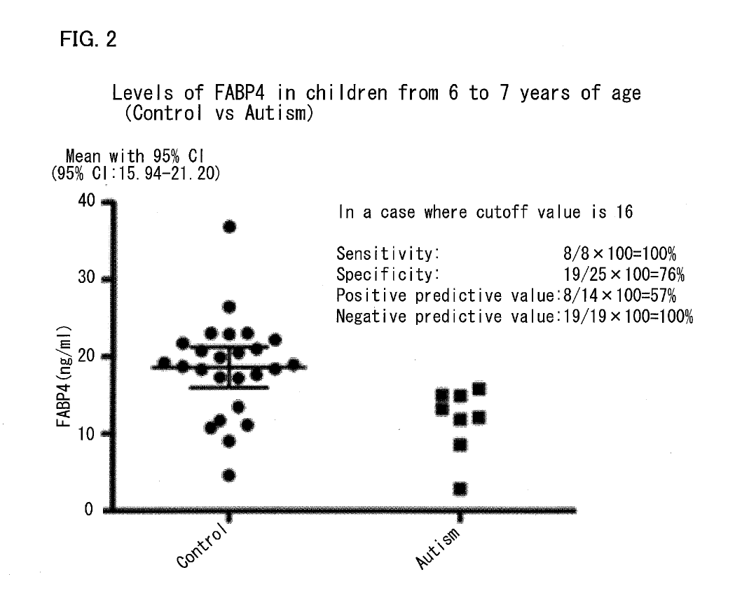

As shown in FIG. 1, it was found that the mean values of fatty acid binding protein FABP4 concentrations in the groups of autistic children 7 years old or younger are significantly lower than the mean value of the control groups of the same age. For more detailed analysis of the groups of children from 6 to 7 years of age, a scatter graph of comparison between the control group and the autistic children group (Control vs Autism) was created regarding the values of fatty acid binding protein FABP4 concentrations in individuals in the groups of children from 6 to 7 years of age, and a Mann-Whitney test was conducted. The cutoff value was a fatty acid binding protein FABP4 concentration of 16 ng/ml. The sensitivity, specificity, positive predictive value, and negative predictive value were calculated by counting the numbers of children. The results are shown in FIG. 2.

(Results of the Analysis)

In the age groups from 6 to 7 years of age, as is evident from the results shown in FIGS. 1 and 2, the autistic children group was remarkably lower in fatty acid binding protein FABP4 concentration than the control group, and there was a significant difference between the groups. Therefore, it was concluded that a value of fatty acid binding protein FABP4 concentration in peripheral blood is useful as an early detection marker for autistic spectrum disorder.

### Example 2

### Preparation of Biological Samples

A test was done in the same manner as that in which the test was done in Example 1, except that it was done on an autistic children group (Autism (native)) of 152 children (including 11 females) from 4 to 18 years of age diagnosed by doctors as having developed autism and a normally-developing children group (control, i.e. a healthy control group) of 119 healthy children (including 27 females) from 4 to 18 years of age.

(Measurement of the Concentration of the Fatty Acid Binding Protein FABP4)

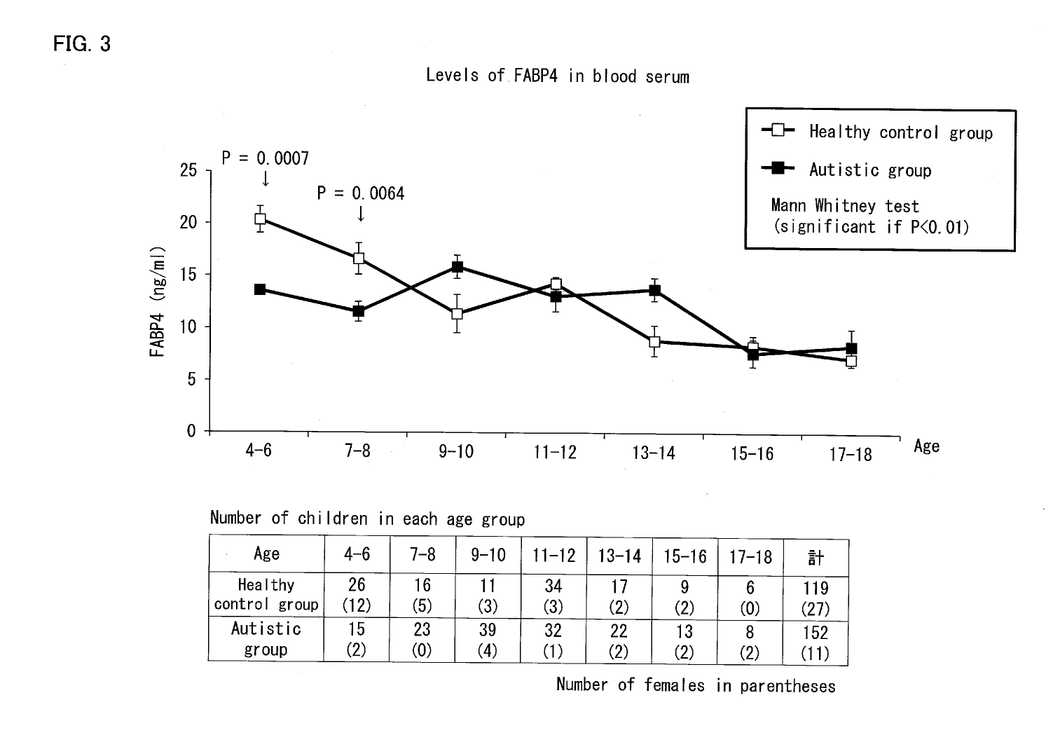

The concentration of the fatty acid binding protein FABP4 in the blood serum thus obtained was measured by the same measuring method as that employed in Example 1. The measurements were performed as follows: The samples were prepared by diluting 30 μL of blood serum of each subject with a buffer solution (EIA buffer, which accompanied the ELISA kit) at a 10-fold dilution factor. Then, the autistic children group and the control group were both divided into age groups from 4 to 6 years of age, from 7 to 8 years of age, from 9 to 10 years of age, from 11 to 12 years of age, from 13 to 14 years of age, from 15 to 16 years of age, and from 17 to 18 years of age, and the mean value of FABP4 protein concentrations (referred to as “mean concentration”) in each age group was calculated. Furthermore, the mean concentrations in the same-age groups (Age) of autistic children and control group children were compared against each other to see if there is a significant difference between the mean concentration in the control group and the mean concentration in the autistic children group. The results are shown in the upper graph of FIG. 3. The number of children in each age group of autistic children and control group children is shown in the lower table of FIG. 3.

(Analysis of the Measurement Results)

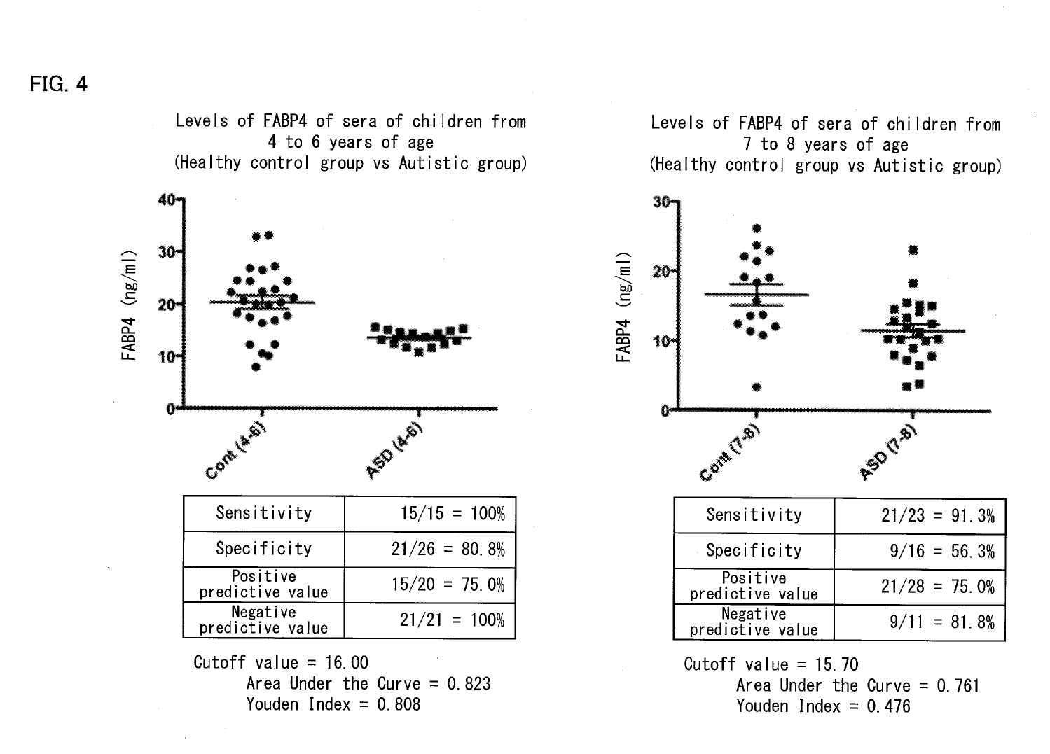

As shown in FIG. 3, it was found that the mean value of fatty acid binding protein FABP4 concentrations in each of the groups of autistic children from 4 to 6 years of age and from 7 to 8 years of age is significantly lower than the mean value of the healthy control groups of the same age. For more detailed analysis of the groups of children from 4 to 6 years of age and from 7 to 8 years of age, scatter graphs of comparison between the control groups and the autistic children groups (Control vs Autism) was created regarding the values of fatty acid binding protein FABP4 concentrations in individuals in the groups of children from 4 to 6 years of age and from 7 to 8 years of age, and a Mann-Whitney test was conducted. ROC (receiver operating characteristic) curves were created. For the age groups from 4 to 6 years of age, the cutoff value was a fatty acid binding protein FABP4 concentration of 16 ng/ml. For the age groups from 7 to 8 years of age, the cutoff value was a fatty acid binding protein FABP4 concentration of 15.7 ng/ml. On the basis of these cutoff values, the sensitivity, specificity, positive predictive value, and negative predictive value were calculated by counting the numbers of positive and negative children. Further, a value of Area Under the Curve and a value of Youden's index were calculated for both the age groups from 4 to 6 years of age and the age groups from 7 to 8 years of age. The results are shown in FIG. 4. Although not illustrated, it was confirmed by a Mann-Whitney test that there is no difference in FABP concentration between male and female individuals in each of the control groups from 4 to 6 years of age and from 7 to 8 years of age. Although not illustrated, it was confirmed by a Mann-Whitney test that there is no difference in BMI (Body Mass Index) between healthy children and autistic children in both the age groups from 4 to 6 years of age and the age groups from 7 to 8 years of age.

(Results of the Analysis)

The upper graphs of FIG. 4 are scatter graphs showing distributions of FABP4 concentrations in blood plasma in the age groups from 4 to 6 years of age and the age groups from 7 to 8 years of age, respectively.

On the basis of the scatter graphs, the numbers of positive and negative children were counted for the presence or absence of development of autism in the autistic groups and the healthy control groups. As a result, all of the 15 children in the autistic group from 4 to 6 years of age were tested positive. On the other hand, 5 out of the 26 children in the healthy control group from 4 to 6 years of age were tested positive, and 21 were tested negative. Further, 21 out of the 23 children in the autistic group from 7 to 8 years of age were tested positive, and 2 were tested negative. On the other hand, 7 out of the 16 children in the healthy control group from 7 to 8 years of age were tested positive, and 9 were tested negative. The lower table of FIG. 2 is a table of sensitivity, specificity, positive predictive value, and negative predictive value as calculated on the basis of these results.

In the age groups from 4 to 6 years of age and from 7 to 8 years of age, as is evident from the results shown in FIGS. 3 and 4, the autistic children groups were remarkably lower in fatty acid binding protein FABP4 concentration than the control groups, and there was a significant difference between the types of groups. Therefore, it was concluded that a value of fatty acid binding protein FABP4 concentration in peripheral blood is useful as an early detection marker for autistic spectrum disorder.

It was demonstrated by the results of Examples 1 and 2 that a value of FABP4 in a child's blood serum can be a diagnostic marker useful for diagnosis of autistic spectrum disorder. Further, since the marker exhibits an especially remarkably significant difference between the autistic groups and healthy control groups of children 8 years or younger, the marker is very useful in early diagnosis.

The present invention is not limited to the description of the embodiments and examples above, but may be altered by a skilled person within the scope of the claims. An embodiment based on a proper combination of technical means disclosed in different embodiments and examples is encompassed in the technical scope of the present invention.

### INDUSTRIAL APPLICABILITY

The present invention is applicable to a test for the presence or absence of a predisposition to autistic spectrum disorder or the presence or absence of development of autistic spectrum disorder.

1. A test method for testing for the presence or absence of a predisposition to autistic spectrum disorder or the presence or absence of development of autistic spectrum disorder,
the test method comprising a test step of determining an amount of at least one of the fatty acid binding proteins FABP3, FABP4, FABP5, and FABP7 in a sample prepared from the living body of a human or determining an expression level of at least one of the FABP3, FABP4, FABP5, and FABP7 genes in the sample.

2. The test method as set forth in claim 1, wherein in the test step, the amount of the fatty acid binding protein FABP4 or the expression level of the FABP4 gene is determined.

3. The test method as set forth in claim 1, wherein the sample is a blood-derived sample.

4. The test method as set forth in claim 1, wherein the amount of the protein being tested is determined.

5. The test method as set forth in claim 1, wherein the human is 8 years old or younger.

6. The test method as set forth in claim 1, wherein in the test step, the presence of a predisposition to autistic spectrum disorder or the presence of development of autistic spectrum disorder is determined when at least one selected from among the amounts of the fatty acid binding proteins FABP3, FABP4, FABP5, and FABP7 in the sample and the expression levels of the FABP3, FABP4, FABP5, and FABP7 genes in the sample is lower than that of a healthy subject.

7. The test method as set forth in claim 1, wherein the autistic spectrum disorder is autism.

8. A test kit for testing for the presence or absence of a predisposition to autistic spectrum disorder or the presence or absence of development of autistic spectrum disorder,
the test kit comprising a nucleic-acid probe, a nucleic-acid primer, a nucleic-acid aptamer, an antibody, or a peptide probe for detecting at least one of the fatty acid binding proteins FABP3, FABP4, FABP5, and FABP7 in a sample prepared from the living body of a human or at least one of expression products of the FABP3, FABP4, FABP5, and FABP7 genes in the sample.


SEQUENCE LISTING (2 sequences)

SEQ ID NO: 1 - protein, 132 aa, Homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 2 - DNA, 838 bp, Homo sapiens
gggtcacagcaccctcctgaaaactgcagcttccttctcaccttgaagaataatcctagaaaactcacaaaatgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccatgatttagcaagcaactaattttctcccaagctgattttattcaatatggttacgttggttaaataaactttttttagatttagaaggtgatgtaatgatgtattcattgtgcttatgatgtattcttagtcataactgagtgaaggaaatgggaaatttgcattatttctttgttctgatatgaataataacatatttcataataattcaaggtaaaaagggatatctatggatttccctaggtaggagataacaagtatgtaccattactgaatat